(19) United States
(12) Plant Patent Application Publication
(10) Pub. No.: US 2002/0152515 P1
(43) Pub. Date: Oct. 17, 2002

(54) ROSE PLANT NAMED 'POULSHRIMP'

(75) Inventors: L. Pernille Olesen, Fredensborg (DK); Mogens N. Olesen, Fredensborg (DK)

Correspondence Address: Poulsen Roser Pacific, Inc., 2550 Old Military Road, Central Point, OR, 97502, US

(73) Assignee: Poulsen Roser ApS.

(21) Appl. No.: 09/780,136

(22) Filed: Feb. 8, 2001

(51) Int. Cl. A01H 5/00

(52) U.S. Cl. PLT/122

(57) ABSTRACT

A new miniature rose which has abundant, orange-red flowers and attractive foliage. This new and distinct variety has shown to be uniform and stable in the resulting generations from asexual propagation.

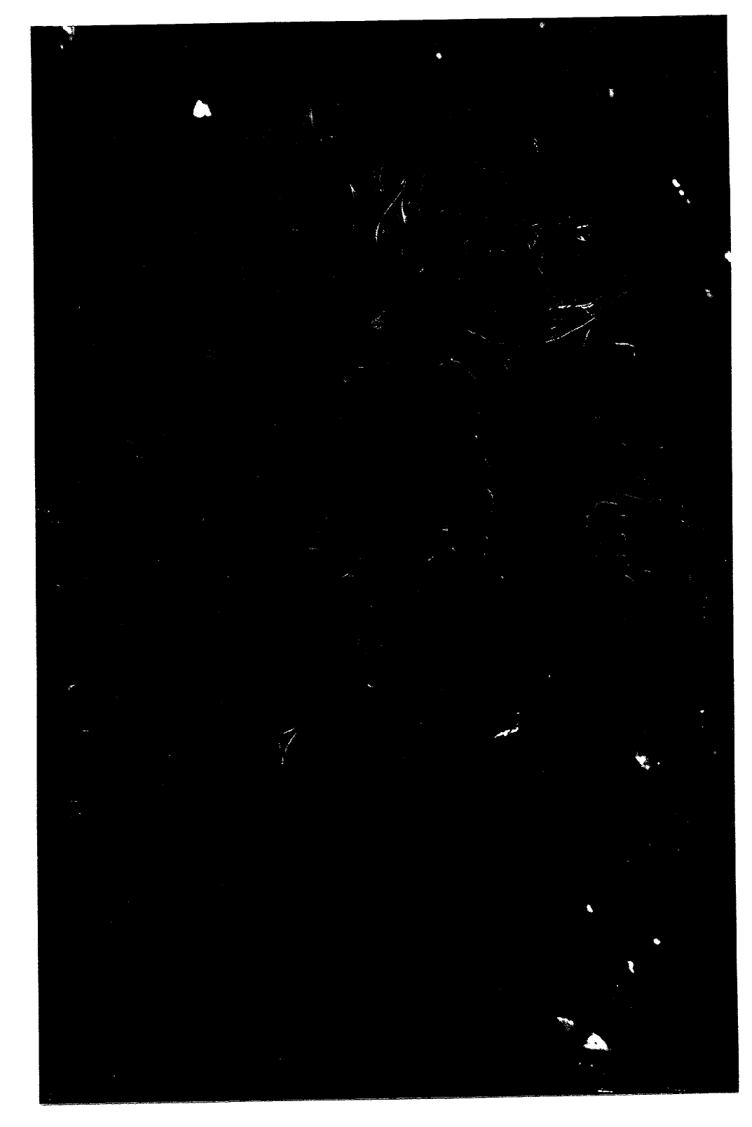

### SUMMARY OF THE INVENTION

The present invention constitutes a new and distinct variety of miniature rose plant which originated from a controlled crossing between ‘POULege’, a rose by the same inventors, described and illustrated in U.S. Plant patent application Ser. 09/282,073 dated Mar. 29, 1999; and ‘POULvic’, a rose by the same inventors, described and illustrated in U.S. Plant Pat. No. 8,012 issued on Oct. 27, 1994. The two parents were crossed during the summer of 1990 and the resulting seeds were planted in a controlled environment in Fredensborg, Denmark. The new variety is named ‘POULshrimp’.

The new rose may be distinguished from its seed parent, ‘POULege’, by the following combination of characteristics:

1. ‘POULshrimp’ has bright orange-red blooms, whereas the ‘POULege’ has salmon-orange blooms.

2. ‘POULshrimp’ is a miniature garden rose, whereas ‘POULege’ is a floribunda rose.

3. ‘POULshrimp’ exhibits more compact and even and growth than ‘POULege’.

The new variety may be distinguished from its pollen parent, ‘POULvic’ by the following combination of characteristics:

1. ‘POULshrimp’ has bright orange-red blooms, whereas ‘POULvic’ has red blooms.

2. ‘POULshrimp’ exhibits less flowers per stem when compared to ‘POULvic’.

3. ‘POULshrimp’ is a larger plant in height and width than ‘POULvic’.

The objective of the hybridization of this rose variety was to create a new and distinct variety for garden use with unique qualities, such as:

1. Uniform and abundant flowers;

2. Vigorous, but compact growth;

3. Disease resistance.

This combination of qualities is not present in previously available commercial cultivars of this type and distinguish ‘POULshrimp’ from all other varieties of which we are aware.

As part of their rose development program, L. Pernille Olesen and Mogens N. Olesen germinated the seeds from the aforementioned hybridization during winter 1990 and conducted evaluations on the resulting seedlings in a controlled environment in Fredensborg, Denmark.

‘POULshrimp’ was selected in the spring of 1991 by the inventors as a single plant from the progeny of the aforementioned hybridization.

Asexual reproduction of ‘POULshrimp’ by traditional budding and rooted cuttings was first done by L. Pernille and Mogens N. Olesen in their nursery in Fredensborg, Denmark in summer 1991. This initial and other subsequent asexual propagations conducted in controlled environments have demonstrated that the characteristics of ‘POULshrimp’ are true to type and are transmitted from one generation to the next.

### BRIEF DESCRIPTION OF THE DRAWING

The accompanying color illustration shows as true as is reasonably possible to obtain in color photographs of this type the variety rose ‘POULshrimp’. Specifically illustrated in SHEET 1 include ‘POULshrimp's’ foliage, flower buds, partially opened buds, and numerous open blooms.

### DETAILED DESCRIPTION OF THE VARIETY

The following is a description of ‘POULshrimp’, as observed in its growth during trials at the testing station Prüfstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULrouge’, a rose variety from the same inventors described and illustrated in U.S. Plant Pat. No. 9,687 issued on Nov. 12, 1996 are compared to ‘POULshrimp’ in Chart 1.

CHART 1

| | ‘POULshrimp’ | ‘POULrouge’ |
|---|---|---|
| Color of open | Red Group 45C | Red Group 40B |
| flower, inner | | |
| side, middle | | |
| zone. | | |
| Petalage | 25-30 | 30-35 |
| Upon opening, | Yellow Group 5C | Yellow Group 7D |
| color basal | | |
| petal spot, | | |
| outer side | | |

Parents:

Seed parent.—‘POULege’.

Pollen parent.—‘POULvic’.

Classification:

Botanical.—Rosa hybrida.

Commercial.—Miniature.

### FLOWER AND FLOWER BUD

Blooming habit: Nearly continuous.

Flower bud:

Bud form.—Broad ovate.

Sepals.—Weak appendages.

Receptacle.—Size: Very small to small.

Peduncle.—Surface: Above average abundance of hairs and prickles.

Borne.—Large tufts with 3-25 flowers per stem.

Flower bloom:

Fragrance.—Little to none.

Size.—Small. Average flower diameter is 50-60 mm when open.

Form.—Shape of flower when viewed from the side: Upper part: Flattened convex. Lower part: Flat. Viewed from above: Star-shaped.

Petalage.—Double. Average range: 20-30 petals under normal conditions.

Color:

Petals.—Inner Side: Middle zone: Red Group 45C. Marginal zone: Red Group 45C. Basal petal spot: Yellow Group 3C. Outer side: Middle zone: Red Group 46D. Marginal zone: Red Group 46D. Basal petal spot: Yellow Group 5C.

General Tonality: Orange-red. On open flower Red Group 46D. No change in the general tonality.

Petals:

Petal reflex.—Strong.

Undulation of margin.—Weak.

Size.—Small.

Reproductive Organs:

Filaments.—Color: Yellow

Hips.—Shape: Funnel-shaped.

### PLANT

Plant growth: Dense, bushy, compact, low, and even in growth. When grown as a budded field grown plant on Rosa multiflora understock, the average height of the plant is 40-50 cm and the average width is 40 cm.

Stems:

Thorns.—Incidence: Few. Size: Small to medium. Shape: Concave to flat.

Plant foliage:

Leaf size.—Small.

Glossy.—Weak.

Color.—Upper Leaf Surface: Dark green Juvenile foliage: Exhibits anthocyanin on young shoots. Anthocyanin intonation: Color: Bronze to reddish brown.

Plant leaves and leaflets:

Leaflet.—Cross Section: Flat. Margin Undulation: Absent to very weak.

Terminal leaflet.—Length of blade: Short. Width of blade: Narrow. Shape: Rounded.

Disease resistance: Above average resistance to mildew, rust, black spot, and Botrytis under normal growing conditions in Fredensborg, Denmark and Hannover, Germany.

Cold Hardiness: The variety ‘POULshrimp’ has been found to be cold hardy in Fredensborg, Denmark; and Hannover, Germany.

1. A new and distinct variety of rose plant of the miniature rose class, substantially as herein illustrated and described as a distinct and novel rose variety due to its abundant orange-red flowers, disease resistance, and extended period of bloom.
